(19) United States
(12) Patent Application Publication
(10) Pub. No.: US 2013/0030183 A1
(43) Pub. Date: Jan. 31, 2013

(54) PROCESS FOR PREPARING A PHARMACEUTICAL COMPOUND

(71) Applicants: Márta Porcs-Makkay, Pomaz (HU); Tamás Gregor, Csomor (HU); Balázs Volk, Budapest (HU); Gábor Németh, Budapest (HU); József Barkóczy, Budapest (HU); Bálint Nyulasi, Papa (HU); Tibor Mezei, Budapest (HU); György Ruzsics, Budapest (HU); Angéla Pandur, Mende (HU); Erika Szilágyi, Tura (HU); Kálmán Nagy, Budapest (HU); Péter Slégel, Budapest (HU); Enikö Molnár, Erd (HU); Jözsef Debreczeni, Budapest (HU)

(72) Inventors: Márta Porcs-Makkay, Pomaz (HU); Tamás Gregor, Csomor (HU); Balázs Volk, Budapest (HU); Gábor Németh, Budapest (HU); József Barkóczy, Budapest (HU); Bálint Nyulasi, Papa (HU); Tibor Mezei, Budapest (HU); György Ruzsics, Budapest (HU); Angéla Pandur, Mende (HU); Erika Szilágyi, Tura (HU); Kálmán Nagy, Budapest (HU); Péter Slégel, Budapest (HU); Enikö Molnár, Erd (HU); Jözsef Debreczeni, Budapest (HU)

(73) Assignee: EGIS GYOGYSZERGYAR NYILVANOSAN MUKODO RESZVENYTARSASAG, Budapest (HU)

(21) Appl. No.: 13/517,339

(22) PCT Filed: Dec. 21, 2010
(86) PCT No.: PCT/HU2010/000148

§ 371 Date: Sep. 28, 2012

(30) Foreign Application Priority Data
Dec. 21, 2009 (HU) P0900795

(51) Int. Cl. C07D 495/04

(52) U.S. Cl. 546/114

(57) ABSTRACT

The object of the present invention is a one-pot process for preparing the 2-acetoxy-5-(2-fluoro-α-cyclopropyl-carbonyl-benzyl)-4,5,6,7-tetrahydro-4H-tieno[3,2-c]-pyridine (prasugrel) of the formula (I) by reacting the 5,6,7,7a-tetrahydro-4H-tieno[3,2-c]-pyridine-2-on of the formula (II) with 2-bromo-1-cyclopropyl-2-(2-fluorophenyl)-etanone of the formula (III) or with 2-chloro-1-cyclopropyl-2-(2-fluorphenyl)-etanone of the formula (IIIa) and acetylating of the formed compound of the formula (IV), wherein the reaction is carried out in the presence of an organic base with an acetylation agent without isolating the compound of the formula (IV). The coupling and acetylation are carried out in the presence of the same organic base such as triethylamine, N,N-diisopropyl-ethylamine or pyridine. At the end of the process the prasugrel of the formula (I) is purified by recrystallization from an organic solvent or a mixture of solvents.

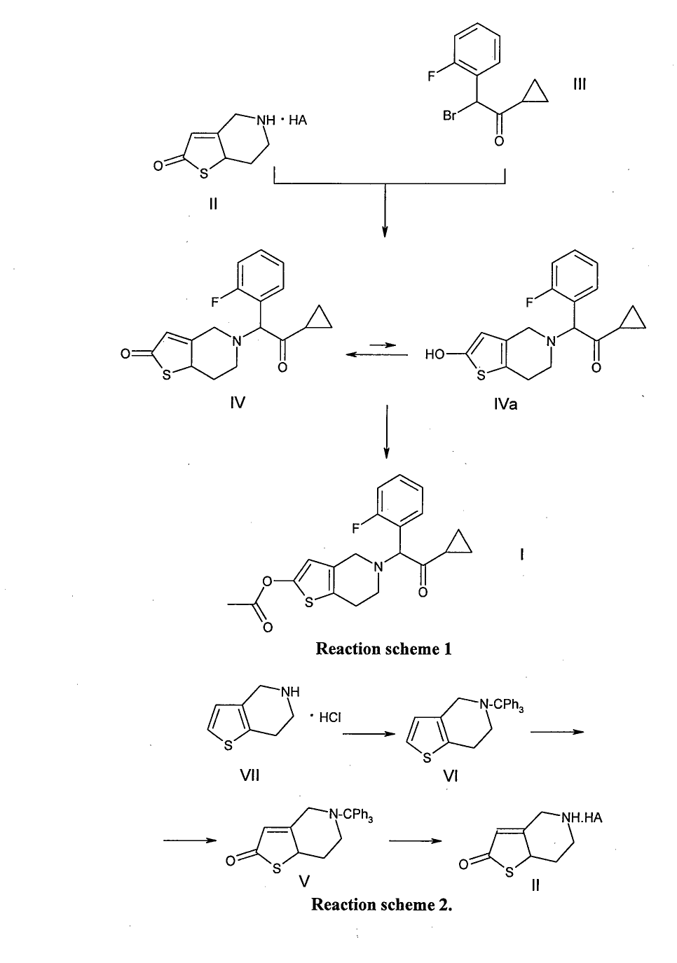

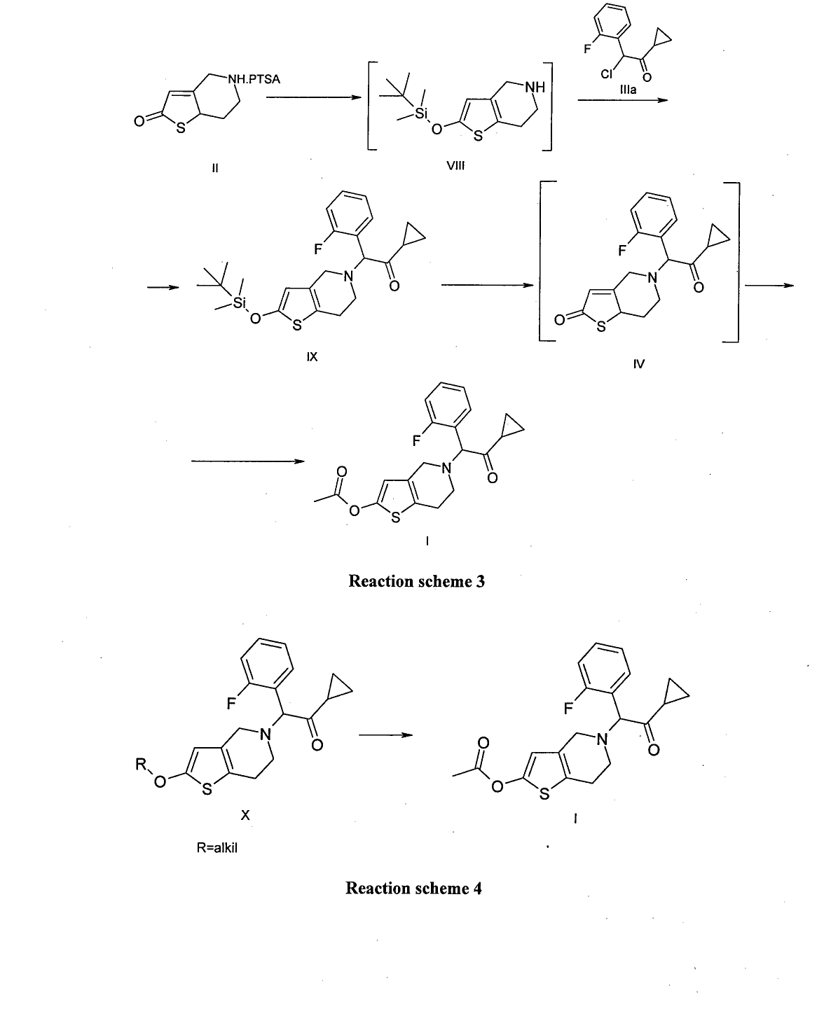

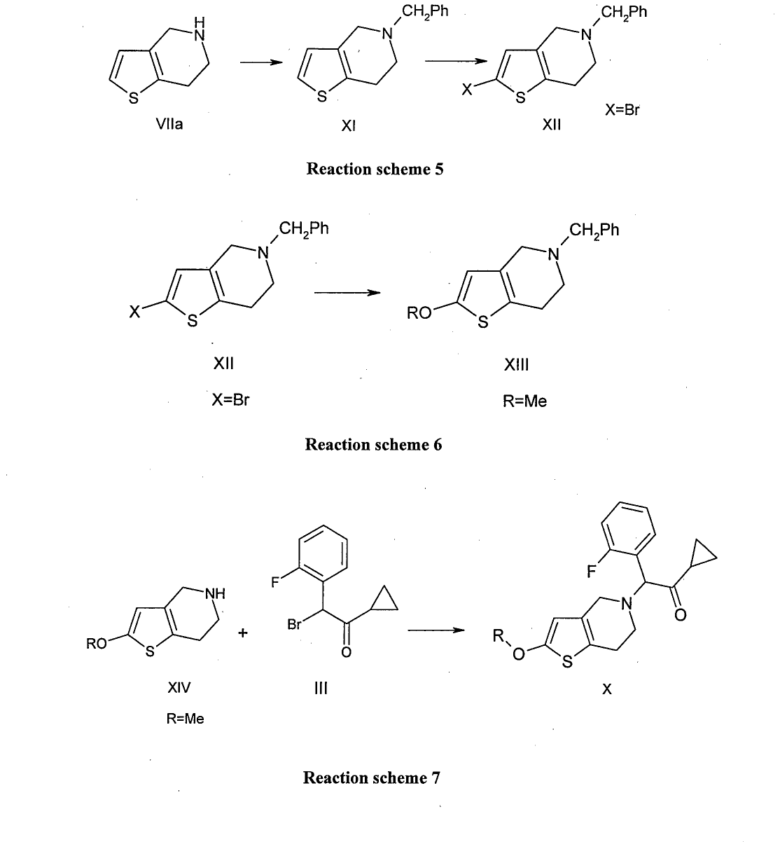

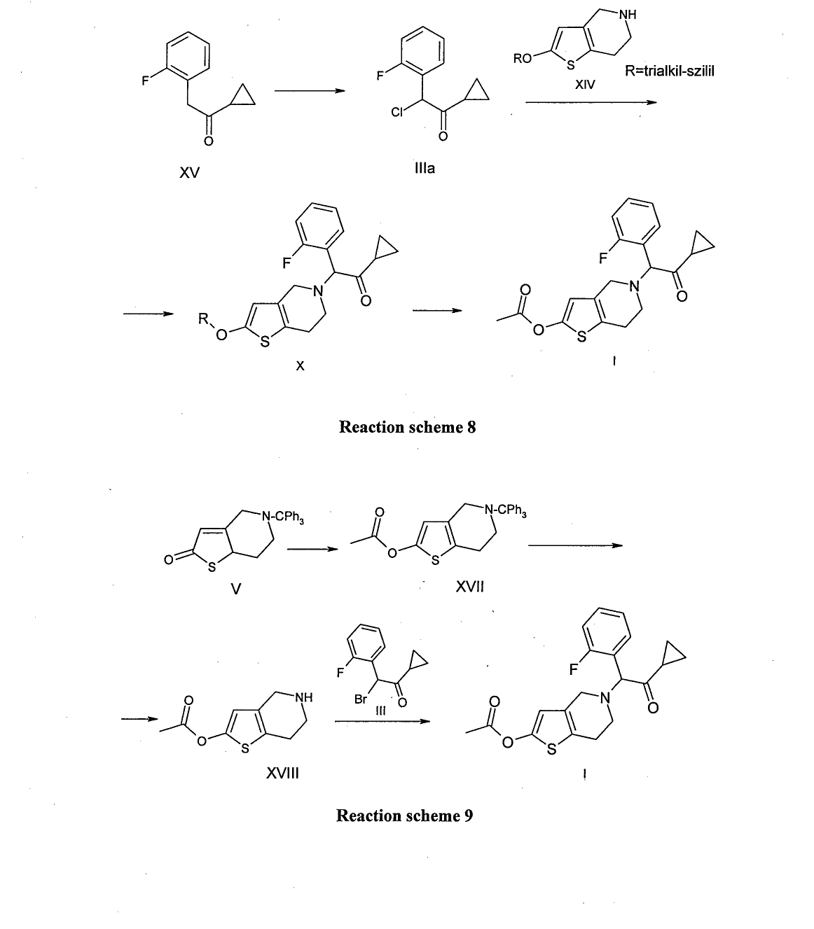

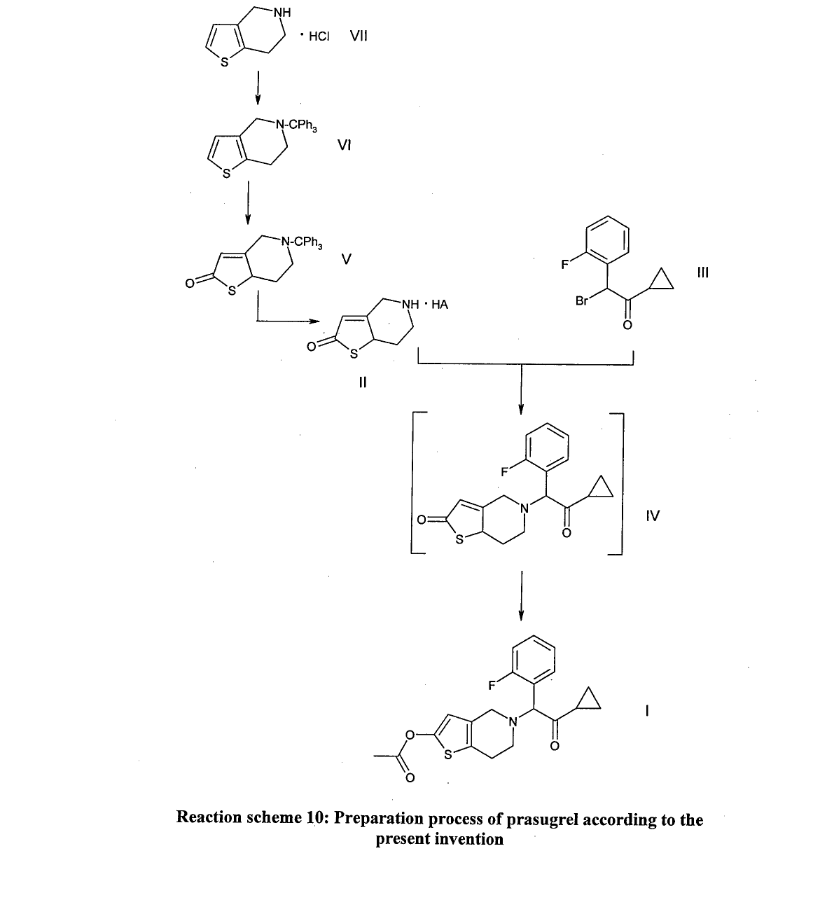

### THE FIELD OF THE INVENTION

The present invention relates to a process for preparing 2-acetoxy-5-(2-fluoro-α-cyclopropyl-carbonyl-benzyl)-4,5,6,7-tetrahydro-4H-tieno[3,2-c]pyridine (prasugrel) of the formula

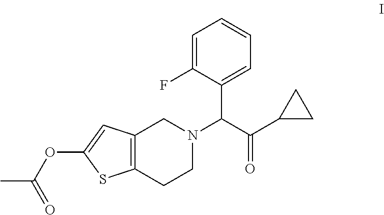 [SMILES: CC(=O)Oc1cc2c(s1)CCN(C(C(=O)C1CC1)c1ccccc1F)C2]

which is advantageously and safely applicable on industrial scale.

2-acetoxy-5-(2-fluoro-α-cyclopropyl-carbonyl-benzyl)-4,5,6,7-tetrahydro-4H-tieno[3,2-c]pyridine (prasugrel) of formula (I) is an important representative of the tetrahydro-tienopyridine derivatives which are used in the pharmaceutical industry as thrombocyte aggregation inhibitors.

### THE STATE OF THE ART

The known synthetic routes for the preparation of prasugrel of the formula (I):

Prasugrel and the analogous compounds and a process for preparing thereof were first described in U.S. Pat. No. 5,288,726 B 1. The preparation process according to U.S. Pat. No. 5,288,726 B1 is shown in reaction scheme 1. The 5,6,7,7a-tetrahydro-4H-tieno[3,2-c]pyridine-2-on hydrochloride of the formula

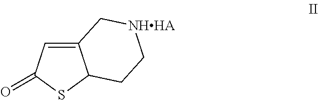

wherein HA stands for HCl—is reacted with 2-bromo-1-cyclopropyl-2-(2-fluorophenyl)-ethanon of the formula

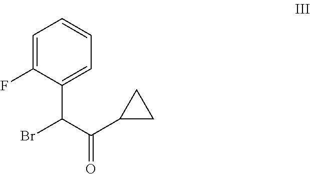 [SMILES: O=C(C1CC1)C(Br)c1ccccc1F]

in dimethyl-formamide at room temperature for 5 hours in the presence of anhydrous potassium carbonate (molar ratio: 1.0:1.0:2.2). The crude product is purified by column chromatography, the yield is 32%. The product is the brown and oily 5-[2-cyclopropyl-1-(2-fluorophenyl)-2-oxoethyl]-5,6,7,7a-tetrahydro-4H-tieno[3,2-c]pyridine-2-on of the formula

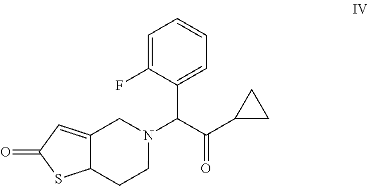 [SMILES: O=C1C=C2CN(C(C(=O)C3CC3)c3ccccc3F)CCC2S1]

mixed with the corresponding 2-hydroxy tautomer. The oily product is crystallized from diisopropyl ether and the yield is not disclosed. As the last step of the synthesis sodium hydride is added to the mixture of the formula (IV) and the solution of dimethyl-formamide and acetic anhydride. The reaction mixture is stirred for 3 hours at room temperature before processing. The crude product is purified by column chromatography. After evaporation the oily product is crystallized form diisopropyl ether, the process yields 65% of prasugrel base, calculated on the intermediate compound of formula (IV) and is very low, 21% calculated on the compound of formula (II)

The preparation of the starting material of the 5,6,7,7a-tetrahydro-4H-tieno[3,2-c]pyridine-2-on hydrochloride of the formula (II)—wherein HA stands for HCl—is not specified in U.S. Pat. No. 5,288,726 B 1, the description only cites known processes without any details. The cited documents (M. Podesta et al., Eur. J. Med. Chem.-Chim. Ther. 9 (5), 487-490 (1974); and Japanese Patent Kokai Application No. Sho 61-246186) do not disclose any preparation process of the key intermediate compound of formula (II) (HA=Cl). Several further applications cite the synthetic route according to reaction scheme 2.

The disadvantage of the process disclosed by U.S. Pat. No. 5,288,726 B1 is that the compound of the formula (IV) and prasugrel of the formula (I) are obtained from the reaction mixture by column chromatography. It is known that column chromatography requires high amount of solvent, therefore scaling up is difficult and in the present case results in a low yield. Another disadvantage of the above process is using sodium-hydride by the acylation step of the preparation of the end-product. The use of sodium-hydride on industrial processes is dangerous and should comply with strict safety prescriptions. A further disadvantage of using sodium-hydride is the processing of paraffin which is used for the suspending step.

The process which is disclosed in EP 1 098 132 B1 is similar to the process described in the basic patent. The 5,6,7,7a-tetrahydro-4H-tieno[3,2-c]pyridine-2-on hydrochloride of the formula (II) (HA=HCl) is reacted with 2-bromo-1-cyclopropyl-2-(2-fluorphenyl)-ethanon of the Formula (III) in dimethyl formamide at room temperature, but differently from the basic patent potassium hydrogen carbonate is used as a base and also a different molar ratio (1.0:0.93:2.8) is used. The reaction mixture is stirred for 2 hours at room temperature, the product is distributed between water and ethyl acetate and after evaporation the product is purified by chromatography. It is crystallized from diisopropyl ether. Thus the yield of intermediate compound of the Formula (IV) is 35%. Acetylation is similar to that described in the basic patent but a mixture of toluene and ethyl acetate in a ratio of 3 to 1 is used instead of 100 to 3 by chromatography as eluent. The yield of the last step is also 65% and the yield of the two steps together is only 23%.

In WO2007/115305 A1 a basically identical process to that according to the basic patent is described. The disclosed processes are based on the same disadvantageous steps as the basic patent and result in similarly low yields.

According to the process described in U.S. Pat. No. 5,874,581 B1 prasugrel of the Formula (I) is produced starting from 5,6,7,7a-tetrahydro-4H-tieno[3,2-c]pyridine-2-on toluene-4-sulfonate of Formula (II) (HA=p-toluene sulfonate, PTSA), wherein the carbonyl group in position 2 is silylated, and the protected intermediate compound of the formula

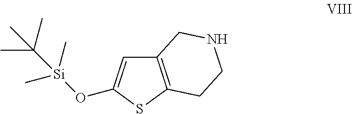 [SMILES: CC(C)(C)[Si](C)(C)Oc1cc2c(s1)CCNC2]

is linked with an appropriate ketone halogenide and the removal of protective group and O-acetylation of the in situ formed oxo intermediate compound of the formula (IV) are carried out in one step. The process is shown in the reaction scheme 3.

The detailed process is the following: The toluene-4-sulphonate salt of the formula (II) is reacted with terc-butyl-dimethyl-chlorosilane in the presence of triethyl amine in dichloro methane solvent for 3 hours at room temperature and the 2-(terc-butyl-dimethylesilyloxy)-4,5,6,7-tetrahydrotieno[3,2-c]pyridine toluene-4-szulfonate is formed. The formed compound is further reacted with 2-chloro-1-cyclopropyl-2-(2-fluorophenyl)-ethanon of the formula

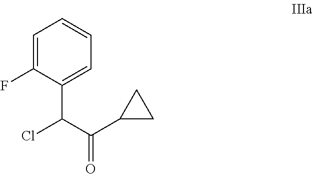 [SMILES: O=C(C1CC1)C(Cl)c1ccccc1F]

without isolation in the presence of sodium iodide, by adding further triethyl amine, at 45° C. and stirred for 12 hours. After processing the reaction mixture the 2-(terc-butyl-dimethylsilyloxy)-5-(α-cyclopropylcarbonile-2-fluorobenzyl)-4,5,6,7-tetrahydrotieno[3,2-c]pyridine of the formula

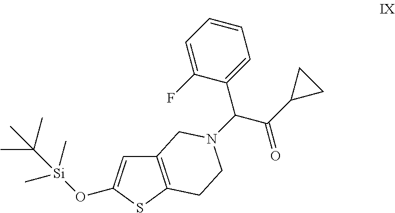 [SMILES: CC(C)(C)[Si](C)(C)Oc1cc2c(s1)CCN(C(C(=O)C1CC1)c1ccccc1F)C2]

is crystallized from acetonitrile. The yield is 73.3%. The obtained silyl derivative is desilylated in THF in the presence of triethyl amine and 4-dimethyleamino-pyridine and it is acetylated with acetic anhydride stirred for 4.5 hours at room temperature without isolating the intermediate compound. The yield of the prasugrel base is 91% calculated on the last reaction step. The yield calculated on toluene-4-sulphonate salt of the formula (II) (HA=PTSA) is 66.7%. The process according to U.S. Pat. No. 5,874,581 B1 the terc-butyl-dimethylsilyloxy group is successfully used for protecting the 2-oxo group to reduce the by-products substituted on the oxo group. Thus the yield calculated on the toluene-4-sulphonate salt of the formula (II) (HA=PTSA) is better than using the process described in the basic patent. Furthermore preferable and simpler method is used to obtain the product than the column chromatography. However the disadvantage of the process described in U.S. Pat. No. 5,874,581 B1 is the introduction of two further process steps. Therefore the synthetic route is expensive and the industrial applicability is difficult because of the use of the water sensible terc-butyl-dimethylsilane and the toxic 4-dimethylamino-pyridine.

The CN 101250192A, CN 101245072A, CN 101245073A and CN 101250193A describe the same novel synthetic process of prasugrel with different process details of certain intermediate compounds.

The CN 101250192A describes the preparation of the prasugrel base from the intermediate compound of the formula

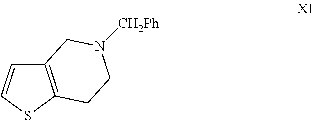

wherein the oxo-group is protected by alkyl group. The process is shown on the reaction scheme 4. The protective group is removed by using acidic and mild reaction medium. The advantage of the process is that it does not use low temperature, flammable or explosive reagents and the process results high yield.

According to the CN 101245072A (Chinese Application) by benzylation of the 4,5,6,7-tetrahydro-tieno[3,2-c]pyridine of the formula

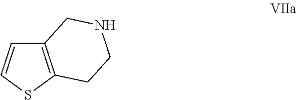

the yield of the intermediate compound of the formula (XI) is 98.7%. The 2-bromo-N-benzyle intermediate compound of the formula

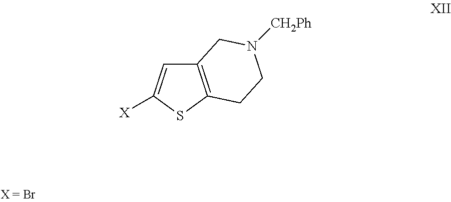

is obtained by bromination of the compound of the formula (IX) with the yield of 97.8% according to the reaction scheme 5. The compound of the formula (XII) is converted to the prasugrel of the formula (I) in several further steps.

CN 101245073A discloses an improved variant of the process described in CN 101245072A, wherein the bromo atom of the 2-bromo derivative of the formula (XII) is substituted by alkoxy group (preferably methoxy group) with sodium-methylate and the yield is 82.2%. The obtained 2-alkioxy intermediate compound of the formula

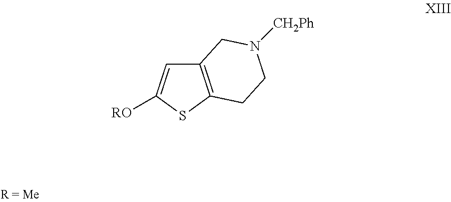

is converted to the prasugrel of the formula (I) in several further steps according to the reaction scheme 6.

In CN 101250193A the 2-alkoxy intermediate compound is formed by linking the 2-alkoxy-4,5,6,7-tetrahydro-tieno[3,2-c]pyridine of the formula

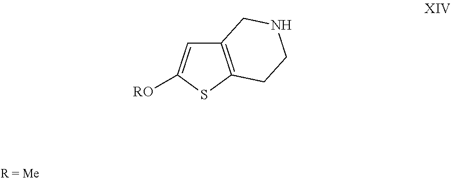

with 2-bromo-1-cyclopropyl-2-(2-fluorphenyl)-ethanone of the formula (III) in the presence of Cu(I) salt and iodine salt (reaction scheme 7.) The advantage of the process is that they do not use low temperature, flammable or explosive reagents and the process results high yield

The disadvantages of the processes described in CN 101250192A, CN 101245072A, CN 101245073A and CN 101250193A are that the economical processing of the dealkylation step of the alkoxy group (preferably demethylation of the methoxy group) is difficult. Further disadvantage is that the formed thiophene compound in the course of hydrogenation of the protective benzyl group on the nitrogen atom is able to act as a catalytic poison.

WO2008/108291 discloses a process for the preparation of prasugrel hydrochloride in which a decreased amount of the impurity of 3-chloro-propyl is formed by ring opening while chlorination of the cyclopropyl ring of prasugrel. The 1-cyclopropyl-2-(2-fluorphenyl)-ethanone of the formula (XV)

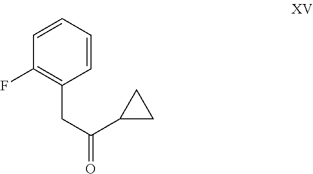 [SMILES: O=C(Cc1ccccc1F)C1CC1]

is chlorinated in position 2 at low temperature and the obtained intermediate compound of the formula (IIIa) is linked with 5,6,7,7a-tetrahydro-4H-tieno[3,2-c]pyridine-2-on derivative of the formula (XIV, R=trialkyl-silyl) which is protected on the oxygen atom (reaction scheme 8.)

EP 2 003 136 A1 describes the process for preparing high purity prasugrel base and acid addition salts thereof (preferably hydrochloride), with reduced content of the desacetyl impurity of the formula (IV). The process disclosed in WO96/11203 is used for producing prasugrel via salt formation and purification of the base. 2-chloro-1-cyclopropyl-2-(2-fluorophenyl)-ethanon of the formula (IIIa) is used by linking, which is formed by halogenation of the appropriate keton of the formula (XV) with chlorine gas, with the yield of 80%. The high purity prasugrel base is recrystallized. Several solvents preferably acetonitrile are mentioned for recrystallization. The synthetic routes and the intermediate compounds are known, which are used in the process. The disadvantage of the present process is using chlorine gas, which is poisonous, difficult to handle and dispose.

WO2009/006859 describes a process, wherein the 5,6,7,7a-tetrahydro-4H-tieno[3,2-c]pyridine-2-on salt of the formula (II) is linked with the appropriate 2-methoxy derivative of the formula

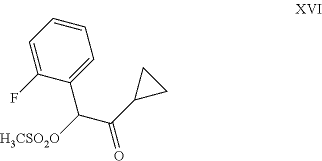

instead of the 2-bromo-1-cyclopropyl-2-(2-fluorphenyl)-ethanon of the formula (III). After preparation of the intermediate compound of the formula (IV) with column chromatography, the yield is 23.7% according to one of the versions and 65.4% according to the other one. The intermediate compound of the formula (XVI) is prepared from 2-fluoro-benzaldehyde and trimethylsilyl-cyanide in several steps, using expensive reagents and the yield is 38.5%. The crude, oily compound of the formula (II) is obtained after acylation and subsequent column chromatography and the crystalline compound is obtained by crystallization from diethyl ether, wherein the yield is 29.2%. The process is not economical and the final product is obtained in each version by column chromatography. The description does not disclose any data about the impurity profile of the final product.

WO2009/062044 discloses two synthetic routes for preparing prasugrel. One of the routes yields 4.6% calculated on the 4,5,6,7-tetrahydro-tieno[3,2-c]pyridine hydrochloride of the formula (VII) or 3.7% considering also the recrystallization step by using the process described in the basic patent with little modification. The other route is shown in the reaction scheme 9. The 5-trityl-5,6,7,7a-tetrahydro-4H-tieno[3,2-c]pyridine-2-on of the formula

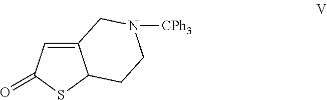

is acetylated and the compound of the formula

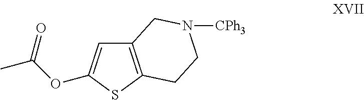

is formed and the protecting group is removed form the nitrogen atom. The thus formed derivative of the formula

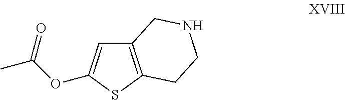

is then linked with the bromo-ketone of the formula (III). The yield is 4.1% calculated on the 4,5,6,7-tetrahydro-tieno[3,2-c]pyridine hydrochloride of the formula (VII) or 3.1% considering also the recrystallization step. It is lower than in the previous process variant.

WO2009/066326 describes an improved and up-scaled process of the basic patent. The 5,6,7,7a-tetrahydro-4H-tieno[3,2-c]pyridine-2-on salt of the formula (II) and the 2-bromo-1-cyclopropyl-2-(2-fluorphenyl)-ethanon of the formula (III) is linked in the presence of potassium carbonate. The formed compound of the formula (IV) is prepared in oily form and is acetylated in the presence of the acid binder diisopropyl-ethyl-amine (DIPEA). The highest yield calculated on the 4,5,6,7-tetrahydro-tieno[3,2-c]pyridine hydrochloride of the formula (VII) is 19.3 and 13%, if we consider the final purification step. Further disadvantages of the process are the use of two different bases in two steps and isolation of the intermediate compound of the formula (VII) is unnecessary, thus economizing manpower and solvent costs.

Known purification processes of prasugrel are the following:

According to U.S. Pat. No. 5,288,726 B1 the crude prasugrel is obtained by column chromatography as an oily product. After recrystallization from diisopropyl ether crystalline prasugrel is obtained. The final product is characterized by melting point and ¹H-NMR spectra.

EP 2 003 136 A1 claims high purity prasugrel, which is obtained by recrystallization from any organic solvent. Suitable solvents are aliphatic hydrocarbons, such as hexane, cyclohexane, heptane, petrolether; aromatic hydrocarbons, such as benzene, toluene, xylene; halogenated hydrocarbons, such as dichloro methane, chloroform, carbon tetrachloride, 1,2-dichloro ethane, chloro benzene, dichloro benzene; ethers, such as diethyl ether, diisopropyl ether, THF, dioxane, dimethoxy ethan, diethylen glycol dimethyl ether; ketones, such as acetone, ethylmethyl ketone, diethyl ketone; esters, such as ethyl-, propyl-, and butyl-acetate; acids, such as acetic acid, propionic acid; nitriles, such as acetonitrile, propionitrile. The preferable solvent is acetonitrile.

According to WO2009/062044 the prasugrel base is purified by recrystallizing form methanol which resulted a yield of 76.4% and a purity of 99.2% measured by HPLC. The purity of the product does not fulfill the requirements of the Pharmacopoeia.

According to WO2009/066326 the prasugrel base is purified by recrystallizing from ethyl acetate-cyclohexane mixture, which resulted in 67.3% yield and the purity improved to 99.8% from 96.5%, measured by HPLC. Another purification method is wherein the prasugrel base is precipitated from its fumarate salt, in ethyl acetate, with aqueous sodium carbonate. After evaporating the organic phase in vacuo, the yield is 52.5%, and to purity of prasugrel is 99.89% measured by HPLC. The yield is 19.3% calculated on 4,5,6,7-tetrahydro-tieno[3,2-c]pyridine hydrochloride of the formula (VII) and 13% when considering the purification step.

### SUMMARY OF THE INVENTION

The object of the present invention is a one-pot process for preparing the 2-acetoxy-5-(2-fluoro-α-cyclopropyl-carbonyl-benzyl)-4,5,6,7-tetrahydro-4H-tieno[3,2-c]-pyridine (prasugrel) of the formula (I) by reacting the 5,6,7,7a-tetrahydro-4H-tieno[3,2-c]-pyridine-2-on of the formula (II) with 2-bromo-1-cyclopropyl-2-(2-fluorophenyl)-etanone of the formula (III) or with 2-chloro-1-cyclopropyl-2-(2-fluorphenyl)-etanone of the formula (IIIa) and acetylating of the formed compound of the formula (IV), wherein the reaction is carried out in the presence of an organic base with an acetylation agent without isolating the compound of the formula (IV). The coupling and acetylation are carried out in the presence of the same organic base and such as triethylamine, N,N-diisopropyl-ethylamine or pyridine. At the end of the process the prasugrel of the formula (I) is purified by recrystallizing from an organic solvent or a mixture of solvents.

### DETAILED DESCRIPTION OF THE INVENTION

The aim of the present invention is to provide an economic, simple synthetic route for producing prasugrel of the formula (I), which does not require column chromatography, is applicable on industrial scale and provides high yield, starts from any salt of 5,6,7,7a-tetrahydro-4H-tieno[3,2-c]pyridine-2-on of the formula (II), preferably the p-toluenesulfonate salt, and run through the intermediate compound 5-[2-cyclopropyl-1-(2-fluorophenyl)-2-oxoethyl]-5,6,7,7a-tetrahydro-4H-tieno[3,2-c]pyridine-2-on of the formula (IV).

The object of the present invention is a one-pot process for producing prasugrel of the formula (I), which starts from any salt of 5,6,7,7a-tetrahydro-4H-tieno[3,2-c]pyridine-2-on of the formula (II), preferably the p-toluenesulfonate salt, and proceeds through the intermediate compound of 5-[2-cyclopropyl-1-(2-fluorofphenyl)-2-oxoethyl]-5,6,7,7a-tetrahydro-4H-tieno[3,2-c]pyridine-2-on of the formula (IV). The process is shown in the reaction scheme 10.

All the known processes are two-step processes for preparing the prasugrel of the formula (I) from the 5,6,7,7a-tetrahydro-4H-tieno[3,2-c]pyridine-2-on p-toluenesulfonate of the formula (II), wherein the 5-[2-cyclopropyl-1-(2-fluorophenyl)-2-oxoethyl]-5,6,7,7a-tetrahydro-4H-tieno[3,2-c]pyridine-2-on intermediate compound of the formula (IV) is isolated. The known processes use two different base at the two steps. Most of the processes use the inflammable sodium hydride during acylation.

It has been surprisingly found that the two processes, which are chemically completely different, can be performed in an one-pot method, thus the process is more economical and requires less manpower than the known processes. The preparation of the intermediate compound of the formula (IV) is difficult, because the compound is capable of the keto-enol tautomery, and the keto form forms diastereomeric mixture, which is difficult to be crystallized due to its mixture character.

The advantage of the process of the present invention is that it can be safely scaled up by replacing the sodium hydroxide by any other organic base. It has been surprisingly found that the two steps can be performed in the same organic solvent (DMF) and in the presence of the same organic base, in spite the fact that the used organic bases are significantly weaker than sodium hydroxide. Any tertiary amines (e.g triethylamine, N,N-diisopropyl-ethylamine, pyridine etc.) can be preferably used as a base instead of the previously used potassium carbonate and hydrogen carbonate (by the linking reaction), and sodium hydride (by acetylation).

The reaction mixture is divided between water-immiscible organic solvent and water and after obtaining from the organic phase, the product is prepared as a crystalline compound. The final product is purified by recrystallizing from organic an solvent, without using column chromatography.

According to the process of the present invention 5,6,7,7a-tetrahydro-4H-tieno[3,2-c]pyridine-2-on p-toluenesulfonate (HA=PTSA) of the formula (II) and 2-bromo-1-cyclopropyl-2-(2-fluorphenyl)-ethanon of the formula (III) are stirred in an organic solvent, (preferably in DMF, THF, toluene, acetonitrile) under adding 1-3 mole equivalents, preferably 2-2.5 mole equivalents of amine, at 20-50° C., preferably 20-30° C., for 1-3, preferably 1-2 hours. The reaction mixture is then divided between water and ethylacetate and the organic phase is dried and evaporated. The residual product is dissolved in organic solvent (preferably in DMF, THF, toluene, acetonitrile) without isolating the crystalline 5-[2-cyclopropyl-1-(2-fluorphenyl)-2-oxoethyl]-5,6,7,7a-tetrahydro-4H-tieno[3,2-c]pyridine-2-on of the formula (IV). 1-2 equivalents, preferably 1-1.5 equivalents of amine and 1-3 equivalents, preferably 1-2 equivalents of acetic acid anhydride is added to the reaction mixture and it is further stirred at 20-50° C., preferably 20-30° C., for 0.5-5 hours, preferably 1-3 hours. The reaction mixture is then divided between water and ethyl acetate and the organic phase is dried and evaporated. The residual product is recrystallized from a suitable organic solvent (acetonitrile, diisopropylether, ethanol), from the mixture of an organic solvent and water or from a mixture of suitable organic solvents (toluene-ethyl acetate, hexane-ethyl acetate).

According to another process variant of the present invention 5,6,7,7a-tetrahydro-4H-tieno[3,2-c]pyridine-2-on p-toluenesulfonate (HA=PTSA) of the formula (II) and 2-bromo-1-cyclopropyl-2-(2-fluorphenyl)-ethanone of the formula (III) are stirred in organic solvent, (preferably in DMF, THF, toluene, acetonitrile) under adding 3-4 mole equivalents of amine, at 20-50° C., preferably 20-30° C., for 1-3, preferably 1-2 hours, then 1-3 equivalents, preferably 1-2 equivalents of acetic acid anhydride are added to the reaction mixture and it is further stirred at 20-50° C., preferably 20-30° C., for 0.5-5 hours, preferably 1-3 hours. The reaction mixture is then divided between water and ethyl acetate and the organic phase is dried and evaporated. The residual product is recrystallized from a suitable organic solvent (acetonitrile, diisopropylether, ethanol), from the mixture of an organic solvent and water or from a mixture of a suitable organic solvents (toluene-ethylacetate, hexane-ethylacetate).

It has been surprisingly found that, contrary to expectations, during the recrystallization of prasugrel of the formula (I) from alcohol (e.g. methanol, ethanol, 2-propanol, preferably ethanol) the transestherification does not occur and the mixture of 5-[2-cyclopropyl-1-(2-fluorophenyl)-2-oxoethyl]-5,6,7,7a-tetrahydro-4H-tieno[3,2-c]pyridine-2-on of the formula (IV) and ethyl acetate is not formed. The amount of the intermediate compound (IV) (which contains 0.3-0.5% diastereomeric mixture) is below 0.03%, therefore the purity of the final product is more than 99.80% measured by HPLC.

The process of the present invention provides prasugrel with a purity of 99.80%, measured by HPLC, total yield 45.7% prasugrel of the formula (I) using the starting compound of the formula (VII), and 46% prasugrel calculated on the intermediate compound of the formula (II). These results show that the process of the present invention gives a significantly higher yield than the known processes. The process of the present invention is readily suitable for industrial scale production and does rot require special or extreme (reaction) conditions and apparatus. During the process of the present invention there is no need to apply low temperature (−78° C.), environmental unfriendly, poisonous, highly corrosive compounds, or high amount of solvents or any technologies difficult for csale-up can be avoided (such as column chromatography). The process according to the present invention is shown in the reaction scheme 10.

The invention is further elucidated by means of following Examples without limiting the scope of protection to the Examples.

### EXAMPLES

### Example 1

### Preparation of the 5-trityl-4,5,6,7-tetrahydro-tieno[3,2-c]pyridine of the formula (VI)

530 cm³ of acetonitrile and 108.0 cm³ (81.8 g; 0.63 mol) of N,N-diisopropyl-ethylamine is added to 52.7 g (0.30 mole) of 4,5,6,7-tetrahydro-tieno[3,2-c]pyridine hydrochloride (VII). While stirring, 87.0 g (0.312 mole) of trityl-chloride are added to the suspension. The mixture is stirred for 3 hours and the precipitated crystals are filtered. The thus obtained intermediate compound can be used in the further synthetic steps without any further purification.

Yield: 108.0 g (94%) colorless, crystalline product

¹H-NMR (DMSO-d₆, 500 MHz): 7.46 (6H, m); 7.31 (6H, m); 7.21 1H, d, J=5.0 Hz); 7.18 (3H, m); 6.72 (1H, d, J=5.0 Hz); 3.28 (2H, s), 2.94 (2H, m); 2.45 (2H, m).

¹³C-NMR (DMSO-d₆, 125 MHz): 142.3; 134.6; 132.9; 128.9; 127.8; 126.3; 125.8; 123.0; 76.8; 47.5; 46.7; 25.9.

Elementary analysis [calculated on the basis of the Formula C₂₆H₂₃NS (M: 381.54)]

Calculated: C 81.85; H 6.08; N 3.67; S 8.40.

Measured: C 81.64; H 6.19; N 3.65; S 8.31.

### Example 2

### Preparation of 5-trityl-5,6,7,7a-tetrahydro-4H-tieno[3,2-c]pyridine-2-on of the formula (V)

750 cm³ of tetrahydrofurane are added to 95.3 g (0.25 mol) of 5-trityl-4,5,6,7-tetrahydro-tieno[3,2-c]pyridine (VI). The solution is cooled to −40° C. and 150 cm³ (0.375 mol) 2.5 M of hexane-butyl-lithium solution are added dropwise at this temperature under argon. The solution is then warmed to +10° C. and stirred for 30 minutes at this temperature. The solution is then cooled to −40° C. and the solution of 86.2 cm³ (0.375 mole) triisopropyl-borate and 200 cm³ THF is dropped in. The solution is then warmed again to +10° C. and stirred for 1 hour at this temperature. The solution is then cooled again to −40° C. and 53.75 cm³ 35 w/w % hydrogen-peroxyde solution are slowly added dropwise. The temperature of the solution is allowed to warm up slowly to room temperature and the solution is stirred for 1 hour at this temperature.

300 cm³ of water are added to the solution while stirring and cooling. The phases are separated and the organic phase dried on MgSO₄ and is evaporated in vacuo. The residual solid product is mixed with heptane. The precipitated crystals are filtered and washed with hexane. The thus obtained product can be used in the further synthetic steps without any further purification.

Yield: 91.4 g (92%) colorless, crystalline product.

Mp.: 194-200° C.

IR (KBr, cm⁻¹): 3442, 3054, 2823, 1681, 1488, 1447, 1096.

¹H-NMR (DMSO-d₆, 500 MHz): 7.46 (6H, m); 7.30 (6H, m); 7.19 (3H, m); 6.07 (1H, s); 4.13 (1H, dd, J=12.1; 2.8 Hz); 3.98 (1H, dd, J=12.1; 6.3 Hz);), 3.34 (1H, dd, J=12.2; 3.2 Hz); 2.40 (1H, m); 2.18 (1H, d, J=12.1 Hz); 2.10 (1H, dd, J=12.2; 3.8 Hz); 1.68 (1H, dt, J=12.1; 1.8 Hz).

¹³C-NMR (DMSO-d₆, 125 MHz): 199.1; 169.8; 129.0; 127.8; 126.5; 125.7; 77.5; 51.6; 50.7; 47.6; 35.2.

Elementary analysis [calculated on the basis of the formula of C₂₆H₂₃NOS (M: 397.54)]

Calculated: C 78.55; H 5.83; N 3.52; S 8.07.

Measured: C 78.15; H 5.50; N 3.31; S 7.70.

### Example 3

### Preparation of 5,6,7,7a-tetrahydro-4H-tieno[3,2-c]-pyridine-2-on toluene-4-sulfonate of the formula II (HA=PTSA)

1300 cm³ of tetrahydrofurane are added to 86.7 g (0.218 mole) of 5-trityl-5,6,7,7a-tetrahydro-4H-tieno[3,2-c]pyridine-2-on (V) and 41.5 g (0.218 mol) of toluene-4-sulfonate-monohydrate are further added under intensive stirring. The reaction mixture is stirred for 2 hours at room temperature. The reaction mixture is cooled in an ice water bath to 0-5° C., stirred for 3-4 hours, filtered and washed with tetrahydrofurane. The thus obtained product can be used in the further synthetic steps without any further purification. Yield: 68.2 g (96%) colorless, crystalline product.

Mp.: 198-200° C.

IR (KBr, cm⁻¹): 3441, 3015, 2827, 1697, 1591, 1446, 1203, 1164, 1123, 1032, 1008.

¹H-NMR (DMSO-d₆, 500 MHz): 9.30 (1H, bs); 8.98 (1H, bs); 7.53 (2H, d, J=8.1 Hz); 7.14 (2H, d, J=8.1 Hz); 6.45 (1H, t, J=1.5 Hz); 4.74 (1H, dd, J=12.1; 5.3 Hz); 4.40 (1H, d, J=13.9 Hz); 4.01 (1H, d, J=13.7 Hz); 3.46 (1H, d, J=11.5 Hz);), 3.28 (1H, t, J=13.0 Hz); 2.59 (1H, m); 2.39 (3H, s); 1.88 (1H, m).

¹³C-NMR (DMSO-d₆, 125 MHz): 197.4; 163.9; 144.9; 138.5; 129.3; 128.5; 125.6; 47.7; 44.0; 42.6; 30.8, 21.0.

Elementary analysis [calculated on the basis of the formula of C₁₄H₁₇NO₄S₂ (M: 327.42)]

Calculated: C 51.36; H 5.23; N 4.28; S 19.59.

Measured: C 51.17; H 5.25; N 4.13; S 19.63.

### Example 4

### Preparation of 2-Acetoxi-5-(2-fluor-α-cyclopropyl-carbonyl-benzyl)-4,5,6,7-tetrahydro-4H-tieno[3,2-c]pyridine (prasugrel, I)

160 cm³ of DMF are added to 65.5 g (0.2 mol) of 5,6,7,7a-tetrahydro-4H-tieno[3,2-c]-pyridine-2-on para-toluenesulfonate (II, HA=PTSA). 75.3 cm³ (56.9 g; 0.44 mol) of N,N-diisopropyl-ethyl-amine (DIPEA) are added to the solution and 55.4 g of 2-bromo-1-cyclopropyl-2-(2-fluorophenyl)-ethanon (III) (containing 92.8% of GC) dissolved is 94 cm³ (88.7 g) of dimethyl-formamide is added dropwise within app. 30 minutes under ice water cooling. The mixture is stirred for 1 hour at room temperature.

37.65 cm³ (28.43 g; 0.22 mol) of DIPEA are added to the reaction mixture and under intensive stirring 28.4 cm³ (30.6 g; 0.30 mol) of acetic acid anhydride are added dropwise. The mixture is stirred for 1 hour at room temperature. The reaction mixture is poured onto the mixture of ice water and ethylacetate. The phases are separated and the aqueous phase is extracted with ethylacetate. The collected organic phases are dried on MgSO₄. The solvent is removed in vacuo and ethanol is added to the remaining product. After cooling to 0-5° C. the precipitated crystals are filtered, washed with ethanol. The yield is 44.7 g (60.0%) crude prasugrel base. The crude product is recrystallized from 5 fold volume ethanol.

Yield: 41.1 g (55.0%) colorless, crystalline product, HPLC purity >99.80%.

Yield for the whole synthetic process, calculated on the 4,5,6,7-tetrahydro-tieno[3,2-c]pyridine hydrochloride of the formula (VII) is 45.7%.

Mp.: 120-121° C.

IR (KBr, cm⁻¹): 3388, 2920, 2767, 1758, 1704, 1586, 1488, 1369, 1217, 1194, 1127, 1011.

¹H-NMR (CDCl₃, 500 MHz): 7.47 (1H, td, J=7.5; 1.8 Hz); 7.30 (1H, m); 7.16 (1H, td, J=7.5; 1.1 Hz); 7.10 (1H, td, J=8.2; 1.1 Hz); 6.26 (1H, s); 4.82 (1H, s); 3.56 (1H, d, J=14.3 Hz); 3.48 (1H, d, J=14.3 Hz); 2.90 (1H, m); 2.78 (3H, m); 2.28 (1H, m); 2.23 (3H, s); 1.05 (1H, m); 1.00 (1H, m); 0.84 (2H, m).

¹³C-NMR (CDCl₃, 125 MHz): 207.4; 167.5; 161.1; 149.4; 130.4; 129.7; 129.3; 125.6; 124.2; 122.0; 115.6; 112.8; 71.5; 50.3; 48.3, 24.9; 20.4; 18.1, 11.8, 11.3.

Elementary analysis [calculated on the basis of the formula of C₂₀H₂₀FNO₃S (M: 373.45)]

Calculated: C 64.33; H 5.40; N 3.75; S 8.59.

Measured: C 64.18; H 5.50; N 3.69; S 8.75.

1. Process for preparing 2-acetoxy-5-(2-fluoro-α-cyclopropyl-carbonyl-benzyl)-4,5,6,7-tetrahydro-4H-tieno[3,2-c]-pyridine (prasugrel) of the formula (I) by reacting 5,6,7,7a-tetrahydro-4H-tieno[3,2-c]-pyridine-2-on of the formula II with 2-bromo-1-cyclopropyl-2-(2-fluorophenyl)-etanone of the formula (III) or with the 2-chloro-1-cyclopropyl-2-(2-fluorphenyl)-etanon of the formula (Ilia) and acetylating of the formed compound of the formula (TV), in the presence of an organic base with an acetylating agent wherein the reaction is carried out in one pot, without isolating the compound of the formula (IV).

2. Process according to the claim 1, wherein the coupling and acetylating reactions are carried out in the presence of the same organic base.

3. Process according to the claim 1, wherein the organic base is triethylamine, N,N-diisopropyl-ethylamine or pyridine.

4. Process according to the claim 1, wherein prasugrel of the formula (I) is purified by recrystallization from an organic solvent or a mixture of solvents.

5. Process according to the claim 4, wherein the organic solvent is ethanol.

6. Process according to the claim 2, wherein the organic base is triethylamine, N,N-diisopropyl-ethylamine or pyridine.
